SEQUENCE LISTING (9 sequences)

SEQ ID NO: 1 - DNA, 27 bp, Artificial Sequence
  Description of Artificial Sequence Synthetic primer
tggaatatttgatgaataaaagtggtt

SEQ ID NO: 2 - DNA, 27 bp, Artificial Sequence
  Description of Artificial Sequence Synthetic primer
tggaatatttgatgaataaaagtggtc

SEQ ID NO: 3 - DNA, 27 bp, Artificial Sequence
  Description of Artificial Sequence Synthetic primer
ggaattggttttttagaataaaggatt

SEQ ID NO: 4 - DNA, 27 bp, Artificial Sequence
  Description of Artificial Sequence Synthetic primer
aaccccctctcattacaacaatactat

SEQ ID NO: 5 - DNA, 430 bp, Homo sapiens
attccggatctggttctccagaacaaaggactttagggcccaaattccgtttattcagtactccaagtcctaaaaacttggaatatctgatgaataaaagtggccgctccccaggctgtctcttgagagaagccaccggcacagctgaccttgcccgctccatcgcgtcactgaccgctcctcagacagatgcgtcaggcatctccggcggccgctccactctgcgccagactcgctgcagcagcggcaggcttcgcacacatccccgcctgagcatgcgcgccagcctgcctctgcggccgcgcaggcgtgcttgtttgccgcagtgcaggggtcccagctccctccctcaccggaatgacctggggggagggggctactggacccctagggccccacagcactgttgcaatgagagggggcctctaga

SEQ ID NO: 6 - DNA, 430 bp, Homo sapiens
attttggatttggttttttagaataaaggattttagggtttaaattttgtttatttagtattttaagttttaaaaatttggaatatttgatgaataaaagtggttgttttttaggttgttttttgagagaagttattggtatagttgattttgtttgttttattgtgttattgattgttttttagatagatgtgttaggtatttttggtggttgttttattttgtgttagatttgttgtagtactggtaggttttgtatatatttttgtttgagtatgtgtgttagtttgtttttgtggttgtgtaggtgtgtttgtttgttgtagtgtaggggttttagtttttttttttattggaatgatttggggggagggggttattggatttttagggttttatagtattgttgtaatgagagggggtttttaga

SEQ ID NO: 7 - DNA, 430 bp, Homo sapiens
atttcggatttggttttttagaataaaggattttagggtttaaatttcgtttatttagtattttaagttttaaaaatttggaatatttgatgaataaaagtggtcgttttttaggttgttttttgagagaagttatcggtatagttgattttgttcgttttatcgcgttattgatcgttttttagatagatgcgttaggtattttcggcggtcgttttattttgcgttagattcgttgtagtagcggtaggtttcgtatatattttcgtttgagtatgcgcgttagtttgtttttgcggtcgcgtaggcgtgtttgtttgtcgtagtgtaggggttttagtttttttttttatcggaatgatttggggggagggggttattggatttttagggttttatagtattgttgtaatgagagggggtttttaga

SEQ ID NO: 8 - DNA, 25 bp, Artificial Sequence
  Description of Artificial Sequence Synthetic oligonucleotide
gacattcgcgatcgccgcccgcttt

SEQ ID NO: 9 - DNA, 25 bp, Artificial Sequence
  Description of Artificial Sequence Synthetic oligonucleotide
caccgccagctcggcttcgagttcg

(19) United States
(12) Patent Application Publication
(10) Pub. No.: US 2010/0047778 A1
(43) Pub. Date: Feb. 25, 2010

(54) Methylation Specific Primer Extension Assay for the Detection of Genomic Imprinting Disorders

(75) Inventors: Ruth Koehler, Albany, CA (US); Chris Beard, Concord, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions Diagnostics, Tarrytown, NY (US)

(21) Appl. No.: 11/996,350

(22) PCT Filed: Jul. 21, 2006
(86) PCT No.: PCT/US06/28516

§ 371 Date: Jan. 21, 2008

Related U.S. Application Data
(60) Provisional application No. 60/702,755, filed on Jul. 26, 2005.

(51) Int. Cl. C12Q 1/68; C07H 21/04

(52) U.S. Cl. 435/6; 536/24.33

(57) ABSTRACT

Provided is a method for determining genomic imprinting disorders in a patient based upon methylation specific primer extension in a format amenable to high throughput and multiplex formats. After bisulfite modification of a genomic sample, DNA is amplified and hybridized to discrimination primers specific for a CpG dinucleotide site in the sample. Because no extension products from the discrimination primers are produced from DNA that has a deletion or functional inactivation at the CpG site, the sample may be diagnosed as having a genomic imprinting disorder by way of comparison with a normal sample.

### CROSS-REFERENCE TO RELATED APPLICATIONS

This application claims the benefit under 35 U.S.C. §371 to PCT/US2006/028516, filed on Jul. 21, 2006, which claims priority under 35 U.S.C. §119(e) to U.S. Provisional Patent Application No. 60/702,755, filed on Jul. 26, 2005, both of which are incorporated in their entireties herein.

### TECHNICAL FIELD

The present invention relates generally to assays for detecting genomic imprinting disorders. More specifically, the present invention relates to a methylation specific primer extension (“MSPE”) assay for the detection of genomic imprinting disorders in an accurate format that is amenable to high throughput and multiplex assay formats.

### BACKGROUND OF THE INVENTION

Normal development of an organism progresses through the expression of both maternally and paternally contributed chromosomes. The phenomenon of genomic imprinting results when one of the chromosomes, i.e., either the maternal or the paternal chromosome, contains genes that are transcriptionally inactivated, leaving its complement to contribute all or most of the genetic information. Disruption of the active allele causes loss of gene product and may result in developmental abnormalities. Two neurobehavioral disorders that result from genomic imprinting disorders are Prader-Willi Syndrome (also referred to as “PWS”) and Angelman Syndrome (also referred to as “AS”), both of which occur in approximately 1 in 15,000 births and are characterized by physical and mental development abnormalities.

Prader-Willi Syndrome is characterized by mental retardation, decreased muscle tone, short stature, emotional lability, and an insatiable appetite which can lead to life-threatening obesity. PWS is caused by the absence or loss of function of segment q11.2-q13 on the long arm of the paternally derived chromosome 15 (15q11.2-15q13). In 70-80% of PWS cases, the region is missing due to a deletion; specifically, a deletion from the distal breakpoint (BP3) to one of two proximal breakpoints (BP1 and BP2) as determined by fluorescence in situ hybridization (“FISH”). When the 15q1.2-15q13 segment is missing from or functionally inactive in the maternally derived chromosome, a completely different disease, Angelman Syndrome, arises. Similar to PWS, 70-80% of AS patients are missing the region due to a deletion; specifically, a 3-5 Mb deletion at 15q11.2-15q13 as determined by FISH.

Angelman Syndrome is characterized by mental retardation, abnormal gait, speech impairment, seizures, and an inappropriate happy demeanor that includes frequent laughing, smiling, and excitability. The uncoordinated gait and laughter have caused some people to refer to this disorder as the “happy puppet” syndrome.

One of the genes found in the PWS chromosomal region codes for the small ribonucleoprotein N (“SNRPN”), which is involved in mRNA processing (i.e., an intermediate step between DNA transcription and protein formation). One of the genes found in the AS chromosomal region codes for an ubiquitin-protein ligase gene (“UBE3A”), which is a key part of the cellular protein degradation system. AS is believed to occur when mutations or loss of function in UBE3A disrupts protein break down during brain development.

Methods currently used to diagnose PWS and AS include standard cytogenetics, FISH, microsatellite analysis, RNA analysis, Southern blot tests using restriction fragment length polymorphisms, and DNA methylation analysis by methods such as methylation-specific PCR.

DNA methylation is known to play a role in regulating gene expression during cell development. The epigenetic event of DNA methylation has been associated with the transcriptional silencing of imprinted genes. Because the mechanism of genomic imprinting has been determined to involve DNA methylation, DNA methylation tests are the preferred test to detect deletions, uniparental disomy, and imprinting center mutations.

In PCT Publication No. WO 99/01580 (inventors Nicholls and Saitoh), DNA methylation is described as having the disadvantages of requiring large amounts of DNA and several days of analysis. To limit the use of DNA in the detection of PWS and AS, Nicholls and Saitoh disclose a method for the detection of these imprinting disorders through the use of a combination of methylation-PCR and methylation-anchor-PCR to distinguish between genotypic classes of patients. Under this system, methylation-PCR is used to detect methylated alleles from normal individuals, both alleles from patients with uniparental disomy of the methylated allele, both alleles from patients with imprinting center mutations that result in the methylation of the normally unmethylated allele, and the methylated allele from patients with deletions in the unmethylated allele. Methylation-PCR does not detect either allele from patients with uniparental disomy of the unmethylated allele, deletions in the methylated allele, imprinting mutations that cause the normally methylated allele to be unmethylated. Methylation-anchor-PCR is used to detect unmethylated alleles from normal individuals, both alleles from patients with uniparental disomy of the unmethylated allele, the unmethylated allele from patients with deletion in the methylated allele, and both alleles from patients with imprinting mutations that cause the normally methylated allele to be unmethylated. Methylation-anchor-PCR does not detect either allele from patients with uniparental disomy of the methylated allele, imprinting mutations that result in the methylation of the normally unmethylated allele, and deletions in the unmethylated allele.

For both methylation-PCR and methylation-anchor-PCR, DNA purified from the sample is digested to completion with methylation-sensitive restriction enzyme. For methylation PCR, digestion is followed by DNA amplification with primers designed to flank the restriction site. DNA digested by the methylation-sensitive restriction site is not amplified because the primer pair does not span a contiguous region; thus, DNA from patients with deletions in the methylated allele will not amplify because they are missing the primer annealing sites. For methylation-anchor-PCR, the restriction enzyme used must produce a single-stranded overhang sufficient for subsequent hybridization and ligation of anchor primers. Because only DNA from unmethylated chromosomes acquires the anchor primer, only unmethylated alleles produce amplification products.

In mammals, DNA methylation usually occurs at cytosines located 5′ of guanines, known as CpG dinucleotides. DNA (cytosine-5)-methyltransferase (DNA-Mtase) catalyzes the reaction by adding a methyl group from S-adenosyl-L-methionine to the fifth carbon position of the cytosine. Most cytosines within CpG dinucleotides are methylated in the human genome, but some remain unmethylated in specific GC-rich areas known as CpG islands. CpG islands are typically between 0.2 to 1 kb in length and are located upstream of many housekeeping and tissue-specific genes, but may also extend into gene coding regions.

U.S. Patent Publication No. 2005/0130170 to Harvey et al., U.S. Patent Publication No. 2005/0130172 to Beard et al., and European Publication No. EP 1 544 309 A1, all owned by Bayer Corporation and each incorporated herein in its entirety, describe the use of MSPE to detect methylation sites at one or more CpG sites. The MSPE method uses a bisulfite-containing reagent to modify CpG sites by converting the unmethylated cytosine into uracil while leaving methylated cytosine unmodified. The chemically treated nucleic acids can be amplified by conventional molecular biology techniques such as PCR amplification and the methylation status in the amplified DNA products may be analyzed by primer extension reaction using tagged reverse primers, dNTPs (deoxynucleotide triphosphates), and ddNTP (dideoxynucleotide triphosphates). Preferably, the reverse primers, dNTPs, and ddNTPs that are incorporated into the extension primers are tagged with a detectable label. In these references, the methylation status of the nucleic acids is used as biomarkers to determine the presence or stage of a disease, such as cancer.

Zeschnigk et al., EUR. J. HUM. GENET. 5(2):94-98 (1997), incorporated herein in its entirety, describes a single tube PCR test to analyze allelic methylation differences at exon 1 of the SNRPN gene on the 15q11.2-15q13 chromosomal region; exon 1 of the SNRPN gene was chosen because of the density of differentially methylated CpG dinucleotides when compared to other regions. The single-tube PCR test, using methylation-specific PCR, uses bisulfite modification of DNA as previously described to convert unmethylated, but not methylated cytosine residues to uracil, and PCR primers specific for the maternal and paternal allele of the SNPRN gene. As a result of the modification, opposite DNA strands are no longer complementary making the PCR reaction on the DNA strand specific. For the initial cycle of the PCR, a primer that anneals to a site downstream of exon 1 of the SNRPN gene, which is identical in both parent alleles, is used to amplify the sense strand. Because the sense strands of the paternal and maternal alleles differ at CpG dinucleotides showing parent-of-origin specific methylation, paternal and maternal specific DNA strands are synthesized. In the second and subsequent PCR cycles, DNA is amplified with a primer binding specifically to the paternal strand and a primer binding specifically to the maternal strand in a duplex PCR reaction sharing the common primer. To distinguish between the two parental alleles, primers are chosen so that the maternal product is 313 bp and the paternal product is 221 bp. The size differential between the two reaction products allows the DNA to be visualized via gel electrophoresis in a pattern that resembles the restriction enzyme pattern from a Southern blot analysis where the maternal methylated allelic fragment is uncut by the restriction enzyme and therefore longer than the unmethylated paternal allelic fragment. Under this system, unreacted DNA is not amplified because the primers do not anneal to unmodified DNA.

The present invention improves upon the DNA methylation techniques presently available in the art for the detection of diseases caused by genomic imprinting by providing a system that does not require large amounts of sample DNA, restriction of the sample DNA with methylation sensitive enzyme, differential hybridization of PCR primers, the preparation of maternal or paternal specific primers, or agarose gel electrophoresis to view PCR products. The present invention improves upon what is known in the art by providing an accurate method of detection of genomic imprinting disorders, such as PWS and AS, through the use of an MSPE assay in combination with discrimination primers, which differ only by one nucleotide at the methylation site, in a format that is amenable to analysis via flow cytometry as well as high throughput and multiplex formats.

### SUMMARY OF THE INVENTION

In one embodiment of the present invention, there is provided a method for detecting a genomic imprinting disorder in a patient comprising the steps of (a) obtaining a sample of genomic DNA from a patient; (b) modifying the genomic DNA in the sample with a compound that converts unmethylated cytosines to uracil while leaving methylated cytosines intact; (c) amplifying the DNA from step (b), wherein the uracil is converted to thymine during initial amplification; (d) hybridizing denatured DNA from step (c) with at least two discrimination primers designed to hybridize to at least one target CpG dinucleotide of the sample, wherein at least one of the discrimination primers has a 3′ cytosine and the other of the at least two discrimination primers has a 3′ thymine; (e) building extension products from the discrimination primers using DNA polymerase in the presence of labeled dNTPs; and (f) identifying the DNA based upon signals emitted from the labeled dNTPs, wherein the genomic imprinting disorder is determined by measuring percentage of methylated or unmethylated extension products to total extension products as determined by the dNTP signals.

In another embodiment of the present invention, there is provided a method for detecting a genomic imprinting disorder in a patient comprising the steps of: (a) obtaining a sample of genomic DNA from a patient; (b) modifying the genomic DNA in the sample with a compound that converts unmethylated cytosines to uracil while leaving methylated cytosines intact; (c) amplifying the DNA from step (b), wherein the uracil is converted to thymine during initial amplification; (d) hybridizing denatured DNA from step (c) with at least two discrimination primers designed to hybridize to at least one target CpG dinucleotide of the sample, wherein at least one of the discrimination primers has a 3′ cytosine and the other of the at least two discrimination primers has a 3′ thymine and further wherein each of the at least two discrimination primers is coupled to a different 5′ ZipCode sequence; (e) building extension products from the discrimination primers using DNA polymerase in the presence of labeled dNTPs; (f) hybridizing the extension product of step (e) to complementary ZipCode (cZipCode) sequences coupled to colored substrates, wherein each unique cZipCode sequence is coupled to a different colored substrate; and (g) identifying the DNA based upon signals emitted from the labeled dNTPs and the colored substrates, wherein the genomic imprinting disorder is detected by measuring percentage of methylated or unmethylated extension products to total extension products as determined by the dNTP signals and methylation status of the extension products is determined by the colored substrate signals.

In a further embodiment of the present invention, there is provided oligonucleotides for use in an MSPE assay comprising at least two discrimination primers complementary to a gene segment of a genomic sample having at least one target CpG site, wherein at least one of the discrimination primers has a 3′ cytosine and the other has a 3′ thymine, wherein extension from the discrimination primer with the 3′ cytosine hybridized to the gene segment indicates a methylated cytosine at the CpG site and extension from the discrimination primer with the 3′ thymine hybridized to the gene segment indicates an unmethylated cytosine at the CpG site.

In yet another embodiment of the invention, there is provided a packaged kit for determining Prader-Willi Syndrome (PWS) or Angelman Syndrome (AS) in a patient, the kit comprising: (a) a compound that when applied to a genomic DNA sample converts unmethylated cytosines to uracil, while leaving methylated cytosines intact; (b) a forward amplification primer and a reverse amplification primer; (c) methylated and unmethylated discrimination primers complementary to a gene segment of a genomic sample with known PWS or AS, wherein both of the discrimination primers are individually coupled to a different 5′ ZipCode sequence, one of the discrimination primers has a 3′ cytosine and the other discrimination primer has a 3′ thymine, and the discrimination primers are designed to hybridize to at least one target CpG dinucleotide of the genomic sample, wherein the at least one CpG dinucleotide is a site associated with PWS or AS; and (d) complementary ZipCode sequences coupled to colored substrates, wherein each unique cZipCode sequence is coupled to a different colored substrate, wherein presence or absence of PWS or AS in the genomic DNA sample is determined by measuring the extension products obtained using the discrimination probes of item (b) and methylation status of the extension products is determined by the colored substrate signals obtained from the cZipCode sequences of item (d).

Additional embodiments, aspects, advantages, and features of the invention will be set forth, in part, in the description that follows, and, in part, will become apparent to those skilled in the art upon examination of the following, or may be learned by practice of the invention.

### BRIEF DESCRIPTION OF THE DRAWINGS

FIG. 1 shows the sequence for the unmodified reverse complement strand of a portion of the SNRPN gene on chromosomal region 15q11.2-15q13 (SEQ ID NO: 5). Within this sequence CpG sites are underlined; sites that are always methylated in PWS and mostly unmethylated in AS are boxed-in; and the target cytosine site, CpG site W, in the 5′ intron of the SNRPN gene is circled.

FIG. 2A shows a reverse complement strand of modified DNA (FIG. 1) that was unmethylated before modification (SEQ ID NO: 6) (upon modification, cytosines in the DNA were converted to uracil resulting in thymine in the reverse complement strand) and FIG. 2B shows a reverse complement strand of modified DNA (FIG. 1) that was methylated before modification (SEQ ID NO. 7) (upon modification, cytosines in the DNA were left intact resulting in cytosine in the reverse complement strand). FIGS. 2A and 2B also show the sequences and locations for the amplification and discrimination primers used for the detection of PWS and/or AS in a sample using the MSPE assay of the present invention. Amplification primers are identified with the shorthand “fPWSuni” (SEQ ID NO: 3) and “rPWSuni,” (SEQ ID NO: 4), which are short for “forward PWS universal primer” and “reverse PWS universal primer,” respectively. Discrimination primers are identified with the shorthand “fPWSunme” (SEQ ID NO: 1) and “fPWSme” (SEQ ID NO: 2), which are short for “forward PWS unmethylated primer” and “forward PWS methylated primer,” respectively. The site investigated for methylation status is the boxed-in “T” at the 3′ end of fPWSunme (FIG. 2A) and the boxed-in “C” at the end of fPWSme (FIG. 2B). The underlined sites in FIGS. 2A and 2B are sites that correspond to the CpG sites of FIG. 1. The boxed-in nucleotides of SEQ ID NOS: 6 and 7 (in FIGS. 2A and 2B) represent nucleotides that have undergone modification.

FIG. 3 shows a schematic of the MSPE assay of the present invention.

FIG. 4 shows data from a patient panel tested for PWS and AS using the MSPE assay of the present invention.

### DETAILED DESCRIPTION OF THE INVENTION

The following definitions and nomenclature are provided to assist in the description of the particular embodiments of the invention, but in no way are intended to be limiting.

As used in this specification and the appended claims, the singular forms “a,” “an,” and “the” include plural referents unless the context clearly dictates otherwise.

As used herein, the term “sample” refers to a biological tissue or fluid obtained from an organism, such as a human. Such samples include, without limitation, blood, plasma, serum, spinal fluid, lymphatic fluid, synovial fluid, cerebrospinal fluid, cell lysates, mucus, tears, saliva, urine, vaginal fluid, amniotic fluid, amniotic cord blood, milk, semen, and secretions of the skin, respiratory, intestinal, or gastrointestinal tract. The samples described herein may be obtained from the organism using conventional techniques such as, for example, through injection with a needle, through biopsy, or through swabbing.

The term “gene” refers to a particular nucleic acid sequence within a DNA molecule that occupies a precise locus on a chromosome and is capable of self-replication by coding for a specific polypeptide chain. The term “genome” refers to a complete set of genes in the chromosomes of each cell of a specific organism.

The term “gene amplification” refers to an increase in the number of copies of a specific gene in an organism's genome. It is understood by one of ordinary skill in the art that the presence of multiple copies of a gene within a genome may result in the production of a corresponding protein at elevated levels.

The term “single nucleotide polymorphisms” or “SNPs” refers to single point variations in genomic DNA.

As used herein, the term “nucleic acid” refers to polynucleotides such as deoxyribonucleic acid (DNA), and, where appropriate, ribonucleic acid (RNA). The term should also be understood to include, as equivalents, analogs of RNA or DNA made from nucleotide analogs, and, as applicable to the embodiment being described, single (sense or antisense) and double-stranded polynucleotides. Expressed Sequence Tags (“ESTs,” i.e., small pieces of DNA sequence usually 200 to 500 nucleotides long generated by sequencing either one or both ends of an expressed gene), chromosomes, cDNAs, mRNAs, and rRNAs are representative examples of molecules that may be referred to as nucleic acids.

The terms “nucleotide” and “nucleoside” refer to nucleosides and nucleotides containing not only the four natural DNA nucleotidic bases, i.e., the purine bases guanine (G) and adenine (A) and the pyrimidine bases cytosine (C) and thymine (T), but also the RNA purine base uracil (U), the non-natural nucleotide bases iso-G and iso-C, universal bases, degenerate bases, and other modified nucleotides and nucleosides. Universal bases are bases that exhibit the ability to replace any of the four normal bases without significantly affecting either melting behavior of the duplexes or the functional biochemical utility of the oligonucleotide. Examples of universal bases include 3-nitropyrrole and 4-, 5-, and 6-nitroindole, and 2-deoxyinosine (dI), that latter considered the only “natural” universal base. While dI can theoretically bind to all of the natural bases, it codes primarily as G. Degenerate bases consist of the pyrimidine derivative 6H,8H-3,4-dihydropyrimido[4,5-c][1,2]oxazin-7-one (P), which when introduced into oligonucleotides base pairs with either G or A, and the purine derivative N6-methoxy-2,6,-diaminopurine (K), which when introduced into oligonucleotides base pairs with either C or T. Examples of the P and K base pairs include P-imino, P-amino, K-imino, and K-amino.

Modifications to nucleotides and nucleosides include, but are not limited to, methylation or acylation of purine or pyrimidine moieties, substitution of a different heterocyclic ring structure for a pyrimidine ring or for one or both rings in the purine ring system, and protection of one or more functionalities, e.g., using a protecting group such as acetyl, difluoroacetyl, trifluoroacetyl, isobutyryl, benzoyl, and the like. Modified nucleosides and nucleotides also include modifications on the sugar moiety, e.g., wherein one or more of the hydroxyl groups are replaced with halide and/or hydrocarbyl substituents (typically aliphatic groups, in the latter case), or are functionalized as ethers, amines, or the like. Examples of modified nucleotides and nucleosides include, but are not limited to, 1-methyladenine, 2-methyladenine, N⁶-methyladenine, N⁶-isopentyl-adenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromo-guanine, 8-chloroguanine, 8-aminoguanine, 8-methylguanine, 8-thioguanine, 5-fluoro-uracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methyl-aminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine, and 2,6-diaminopurine.

As used herein, the term “methylation” refers to the covalent attachment of a methyl group at the C5-position of the nucleotide base cytosine within the CpG dinucleotides of a gene regulatory region. The terms “methylation site” and “methylation status” are used interchangeably to refer to the presence or absence of 5-methylated cytosine at one or a plurality of CpG dinucleotides within a DNA sequence. A methylation site refers to a sequence of contiguous linked nucleotides that is recognized and methylated by a sequence-specific methylase. Furthermore, a methylation site also refers to a specific cytosine of a CpG dinucleotide. A methylase is an enzyme that methylates (i.e., covalently attaches a methyl group to) one or more nucleotides at a methylation site.

Within the context of the present invention, the phrase “a compound that converts unmethylated cytosines to uracil while leaving methylated cytosines intact” is meant to include bisulfite conversion, which is the only currently known method of converting unmethylated cytosines to uracil; however, the term is not meant to be limited to bisulfite conversion, rather, the term is intended to cover any compounds, including chemical compounds and enzymes, that may be found in the future to have the same or similar action to that currently seen with bisulfite. With respect to compounds that convert unmethylated cytosines to uracil, DNA or strands of DNA that have had cytosines converted with a compound such as bisulfite are frequently referred to as “modified” DNA or “modified” strands.

The term “target” refers to molecule, gene, or genome, containing a nucleic acid sequence or sequence segment that is intended to be characterized by way of identification, quantification, or amplification.

As used herein, the term “oligonucleotide” encompasses polydeoxyribonucleotides (containing 2-deoxy-D-ribose), polyribonucleotides (containing D-ribose), any other type of polynucleotide that is an N-glycoside of a purine or pyrimidine base, and other polymers containing normucleotidic backbones (e.g., protein nucleic acids and synthetic sequence-specific nucleic acid polymers commercially available from the Anti-Gene Development Group, Corvallis, Oreg., as NEUGENE™ polymers) or nonstandard linkages, providing that the polymers contain nucleobases in a configuration that allows for base pairing and base stacking, such as is found in DNA and RNA. Thus, “oligonucleotides” herein include double- and single-stranded DNA, as well as double- and single-stranded RNA and DNA:RNA hybrids, and also include known types of modified oligonucleotides, such as, for example, oligonucleotides wherein one or more of the naturally occurring nucleotides is substituted with an analog; oligonucleotides containing internucleotide modifications such as, for example, those with uncharged linkages (e.g., methyl phosphonates, phosphotriesters, phosphoramidates, carbamates, etc.), negatively charged linkages (e.g., phosphorothioates, phosphorodithioates, etc.), and positively charged linkages (e.g., aminoalkylphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), and those containing alkylators. There is no intended distinction in length between the terms “polynucleotide” and “oligonucleotide,” and these terms will be used interchangeably. These terms refer only to the primary structure of the molecule. As used herein the symbols for nucleotides and polynucleotides are according to the IUPAC-IUBMB Joint Commission on Biochemical Nomenclature (see, http://www.chem.qmul.ac.uk/iupac/jcbn).

Oligonucleotides can be synthesized by known methods. Background references that relate generally to methods for synthesizing oligonucleotides include those related to 5′-to-3′ syntheses based on the use of β-cyanoethyl phosphate protecting groups. See, e.g., de Napoli et al., GAZZ CHIM ITAL 114:65 (1984); Rosenthal et al., TETRAHEDRON LETT 24:1691 (1983); Belagaje and Brush, NUC ACIDS RES 10:6295 (1977); in references which describe solution-phase 5′-to-3′ syntheses include Hayatsu and Khorana, J AM CHEM SOC 89:3880 (1957); Gait and Sheppard, NUC ACIDS RES 4: 1135 (1977); Cramer and Koster, ANGEW CHEM INT ED ENGL 7:473 (1968); and Blackburn et al., J CHEM SOC PART C, at 2438 (1967). Additionally, Matteucci and Caruthers, J AM CHEM SOC 103:3185-91 (1981) describes the use of phosphochloridites in the preparation of oligonucleotides; Beaucage and Caruthers, TETRAHEDRON LETT 22:1859-62 (1981), and U.S. Pat. No. 4,415,732 to Caruthers et al. describe the use of phosphoramidites for the preparation of oligonucleotides. Smith, AM BIOTECH LAB, pp. 15-24 (December 1983) describes automated solid-phase oligodeoxyribonucleotide synthesis; and T. Horn and M. S. Urdea, DNA 5:421-25 (1986) describe phosphorylation of solid-supported DNA fragments using bis(cyanoethoxy)-N,N-diisopropylaminophosphine. See also, references cited in Smith, supra; Warner et al., DNA 3:401-11 (1984); and T. Horn and M. S. Urdea, TETRAHEDRON LETT 27:4705-08 (1986).

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C.

The term “universal sequences” refers to sequences that may be used to test many different samples, in other words, universal sequences are independent of the sequences being analyzed.

The term “ZipCode” and “ZipCode sequences” refers to a set of universal sequences with natural or non-natural bases that are used to visualize gene products. For example, a sequence is visualized with a ZipCode when the ZipCode sequence binds to a cZipCode (complementary ZipCode) sequence that is covalently attached to a colored substrate, such as a fluorescent microsphere, which may be visualized using flow cytometry. Generally, the ZipCode sequence is attached to the 5′ end of a primer that is being used to investigate a target site and the cZipCode sequence is attached to the 3′ end of a colored substrate (see Example 2 and SEQ ID NOS: 8 and 9).

The term “flow cytometry” refers to the procedure well known in the art for measuring the physical and chemical characteristics of cells or particles as they travel in suspension one by one past a sensing point. Generally, a flow cytometer consists of a light source, collection optics, and a computer to translate signals to data. In most cytometers, the light source of choice is a laser that emits coherent light at a specified wavelength. Scattered and emitted fluorescent light is collected by two lenses (one set in front of the light source and one set at right angles) and by a series of optics, beam splitters and filters that measure specific bands of fluorescence. Physical characteristics that may be measured using flow cytometry include cell size, shape, internal complexity, and cell components. In order to visualize the cells in the flow cytometer, the cells must be tagged with a fluor, such may be accomplished using the ZipCode procedure described above.

As used herein, the term “probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” refers to an oligonucleotide, whether produced naturally as in a purified restriction digest or produced synthetically, which is capable of acting as a point of initiation of synthesis when placed under conditions in which synthesis of a primer extension product that is complementary to a nucleic acid strand is induced, i.e., in the presence of appropriate nucleotides and an agent for polymerization such as a DNA polymerase in an appropriate buffer and at a suitable temperature. Within the context of the present invention, the term “amplification primer” refers to those primers used in target amplification, such as PCR (which is a geometric amplification reaction), and the term “discrimination primer” refers to those primers used in the MSPE reaction (which is a linear extension reaction).

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation. Typical hybridizing conditions include the use of solutions buffered to a pH from about 7 to about 8.5 and temperatures of from about 30° C. to about 60° C., preferably from about 37° C. to about 55° C. for a time period of from about one second to about one day, preferably from about 15 minutes to about 16 hours, and most preferably from about 15 minutes to about three hours. Hybridization conditions also include an buffer that is compatible, i.e., chemically inert, with respect to primers, probes, and other components, yet still allows for hybridization between complementary base pairs, can be used. The selection of such buffers is within the knowledge of one of ordinary skill in the art.

It is understood by one of ordinary skill in the art that the isolation of DNA and RNA target sequences from a sample requires different hybridization conditions. For example, if the sample is initially disrupted in an alkaline buffer, double stranded DNA is denatured and RNA is destroyed. By contrast, if the sample is harvested in a neutral buffer with SDS and proteinase K, DNA remains double stranded and cannot hybridize with the primers and/or probes and the RNA is protected from degradation.

With respect to the sequences disclosed herein, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because nucleic acids do not require complete complementarity in order to hybridize, the probe and primer sequences disclosed herein may be modified to some extent without loss of utility. Generally, sequences having homology of 80% or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer. Such minor modifications of the disclosed sequences and any necessary adjustments of hybridization conditions to maintain specificity require only routine experimentation and are within the ordinary skill in the art.

The terms “support” and “substrate” are used interchangeably to refer to any solid or semi-solid surface to which an oligonucleotide probe or primer, analyte molecule, or other chemical entity may be anchored. Suitable support materials include, but are not limited to, supports that are typically used for solid phase chemical synthesis such as polymeric materials and plastics for use in beads, sheets, and microtiter wells or plates examples including without limitation, polystyrene, polystyrene latex, polyvinyl chloride, polyvinylidene fluoride, polyvinyl acetate, polyvinyl pyrrolidone, polyacrylonitrile, polyacrylamide, polymethyl methacrylate, polytetrafluoroethylene, polyethylene, polypropylene, polycarbonate, and divinylbenzene styrene-based polymers; polymer gels; agaroses such as SEPHAROSE®; dextrans such as SEPHADEX®); celluloses such as nitrocellulose; cellulosic polymers; polysaccharides; silica and silica-based materials; glass (particularly controlled pore glass) and functionalized glasses; ceramics, and metals. Preferred supports are solid substrates in the form of beads or particles, including microspheres, nanospheres, microparticles, and nanoparticles.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms (particularly ³²P and ¹²⁵I), electron-dense reagents, enzymes, and ligands having specific binding partners. Enzymes are typically detected by their activity. Within the context of flow cytometric analysis, preferred labels include biotinylated primary agents (such as biotinylated dNTPs) that hybridized to a target (such as an amplification sequence from a PCR) and streptavidin-phycoerythrin (“SA-PE”) as secondary agents, where the streptavidin acts as a developer by binding to the biotinylated primary agent and the phycoerythrin acts as the stain.

The term “target amplification” refers to a procedure that amplifies a gene or portion of a gene, such as, for example, PCR or linear amplification (see e.g., Phillips and Eberwine, METHODS 10(3):283-288 (1996)). The terms “amplification sequence” and “amplification product” are used interchangeably to refer to the single-stranded sequences that are the end product of a PCR.

The term “PCR” refers to the polymerase chain reaction, disclosed in U.S. Pat. No. 4,683,195 to Mullis et al. and U.S. Pat. No. 4,683,202 to Mullis, both incorporated herein by reference. Briefly, in the PCR technique, a sample of DNA is mixed in a solution with a molar excess of two oligonucleotide primers of 10-30 base pairs each that are prepared to be complementary to the 3′ end of each strand of the DNA duplex; a molar excess of unattached nucleotide bases (i.e., dNTPs); and DNA polymerase, (preferably Taq polymerase, which is stable to heat), which catalyzes the formation of DNA from the oligonucleotide primers and dNTPs. Of the two primers, one is a forward primer that will bind in the 5′-3′ direction to the 3′ end of one strand of the denatured DNA analyte and the other is a reverse primer that will bind in the 3′-5′ direction to the 3′ end of the other strand of the denatured DNA analyte. The solution is heated to 94-96° C. to denature the double-stranded DNA to single-stranded DNA. When the solution cools, the primers bind to the separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. In other words, an extension product synthesized from the forward primer, upon separation, would serve as a template for a complementary extension product synthesized from the reverse primer. Similarly, the extension product synthesized from the reverse primer, upon separation, would serve as a template for a complementary extension product synthesized from the forward primer. In this way, the region of DNA between the primers is selectively replicated with each repetition of the process. Since the sequence being amplified doubles after each cycle, a theoretical amplification of one billion copies may be attained after repeating the process for a few hours; accordingly, extremely small quantities of DNA may be amplified using PCR in a relatively short period of time.

Where the starting material for the PCR reaction is RNA, complementary DNA (“cDNA”) is made from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. The enzymes are used in genetic engineering to produce specific cDNA molecules from purified preparations of mRNA. A PCR used to amplify RNA products is referred to as reverse transcriptase PCR or “RT-PCR.”

The term “multiplex” refers to multiple assays that are carried out simultaneously, in which detection and analysis steps are generally performed in parallel. As used herein, a multiplex assay may also be termed according to the number of target sites that the assay aims to identify. For example, a multiplex assay that is designed to identify six DNA methylation sites may be referenced a “sixplex” assay and a multiplex assay that is designed to identify eleven DNA methylation sites may be referenced an “elevenplex” assay. Multiplex assays are typically hybridization assays.

The following description of the preferred embodiments and examples are provided by way of explanation and illustration and are not to be viewed as limiting the scope of the invention as defined by the claims. Further, when examples are given, they are intended to be exemplary only and not to be restrictive.

As previously noted, the present invention improves upon what is known in the art by providing an accurate method of detection of genomic imprinting disorders, such as PWS and AS, through the use of an MSPE assay using discrimination primers, which differ only by one nucleotide at a DNA methylation site, in a format that is amenable to high throughput and multiplex formats.

In one embodiment of the present invention, the MSPE assay of the present invention is a method for detecting a genomic imprinting disorder in a patient comprising the steps of (a) obtaining a sample of genomic DNA from a patient; (b) modifying the genomic DNA in the sample with a compound that converts unmethylated cytosines to uracil while leaving methylated cytosines intact; (c) amplifying the DNA from step (b), wherein the uracil is converted to thymine during initial amplification; (d) hybridizing denatured DNA from step (c) with at least two discrimination primers designed to hybridize to at least one target CpG dinucleotide of the sample, wherein at least one of the discrimination primers has a 3′ cytosine and the other of the at least two discrimination primers has a 3′ thymine; (e) building extension products from the discrimination primers using DNA polymerase in the presence of labeled dNTPs; and (f) identifying the DNA based upon signals emitted from the labeled dNTPs, wherein the genomic imprinting disorder is determined by measuring percentage of methylated or unmethylated extension products to total extension products as determined by the dNTP signals.

In one embodiment of the aforementioned MSPE assay, the discrimination primers of step (d) are further individually coupled to different 5′ ZipCode sequences. In this embodiment, prior to step (f), the at least two discrimination primers are hybridized to cZipCode sequences coupled to colored substrates, where each unique cZipCode sequence is coupled to a different colored substrate.

In another embodiment of the present invention, the MSPE assay of the present invention is a method for detecting a genomic imprinting disorder in a patient comprising the steps of (a) obtaining a sample of genomic DNA from a patient; (b) modifying the genomic DNA in the sample with a compound that converts unmethylated cytosines to uracil while leaving methylated cytosines intact; (c) amplifying the DNA from step (b), wherein the uracil is converted to thymine during initial amplification; (d) hybridizing denatured DNA from step (c) with at least two discrimination primers designed to hybridize to at least one target CpG dinucleotide of the sample, wherein at least one of the discrimination primers has a 3′ cytosine and the other of the at least two discrimination primers has a 3′ thymine and further wherein each of the at least two discrimination primers is individually coupled to a different 5′ ZipCode sequence; (e) building extension products from the discrimination primers using DNA polymerase in the presence of labeled dNTPs; (f) hybridizing the DNA of step (e) to cZipCode sequences coupled to colored substrates, wherein each unique cZipCode sequence is coupled to a different colored substrate; and (g) identifying the DNA based upon signals emitted from the labeled dNTPs and the colored substrates, wherein the genomic imprinting disorder is detected by measuring percentage of methylated or unmethylated extension products to total extension products as determined by the dNTP signals and methylation status of the extension products is determined by the colored substrate signals.

In preferred embodiments of the MSPE assays described above, the genomic DNA in the sample is modified with sodium bisulfite; the DNA is amplified by PCR; the labeled dNTPs are biotinylated dNTPs; the extension products from the discrimination primers are visualized upon reaction of the biotinylated dNTPs with SA-PE; and the dNTP signals are measured by flow cytometry. In assays where the ZipCode sequences are coupled to the discrimination primers, it is preferred that the different colored substrates are different colored fluorescent microspheres. Substrate signals from the colored substrates are also measured by flow cytometry. In application, the genomic DNA is preferably from a human patient.

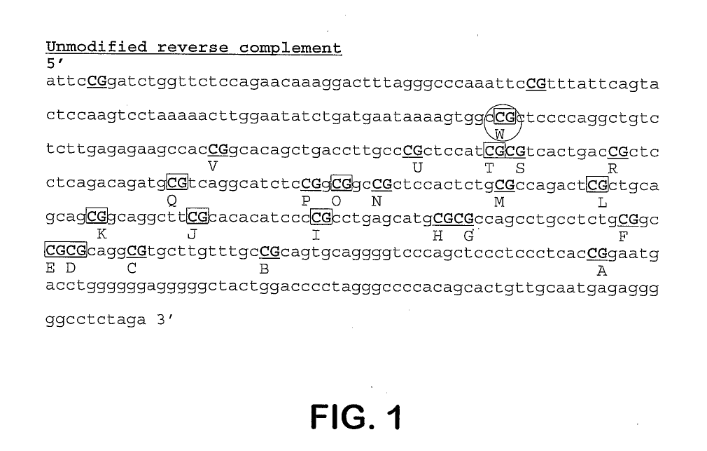

In a preferred embodiment of the present invention, the MSPE assay of the present invention is used to screen a sample for both PWS and AS in a single multiplex assay. Because CpG site W in intron 1 of the unmodified reverse complement strand of the small ribonucleoprotein N(SNRPN) gene on the 15q11.2-15q13 chromosomal region has been found to have good MSPE assay sensitivity and specificity, diagnosis of a patient for PWS or AS may be accomplished by conducting the MSPE assay exclusively on CpG site W; CpG site W is circled in FIG. 1. When CpG site W is used as the target site, the at least two discrimination primers have the sequences of SEQ IDS NOS. 1 and 2.

In a further embodiment of the present invention, there is provided oligonucleotides for use in an MSPE assay comprising at least two discrimination primers complementary to a gene segment of a genomic sample having at least one target CpG site, wherein the gene segment has been modified with a compound that converts unmethylated cytosines to uracil while leaving methylated cytosines in CpG dinucleotides intact, and further wherein at least one of the discrimination primers has a 3′ cytosine and the other has a 3′ thymine, wherein extension from the discrimination primer with the 3′ cytosine hybridized to the gene segment indicates a methylated cytosine at the CpG site and extension from the discrimination primer with the 3′ thymine hybridized to the gene segment indicates an unmethylated cytosine at the CpG site.

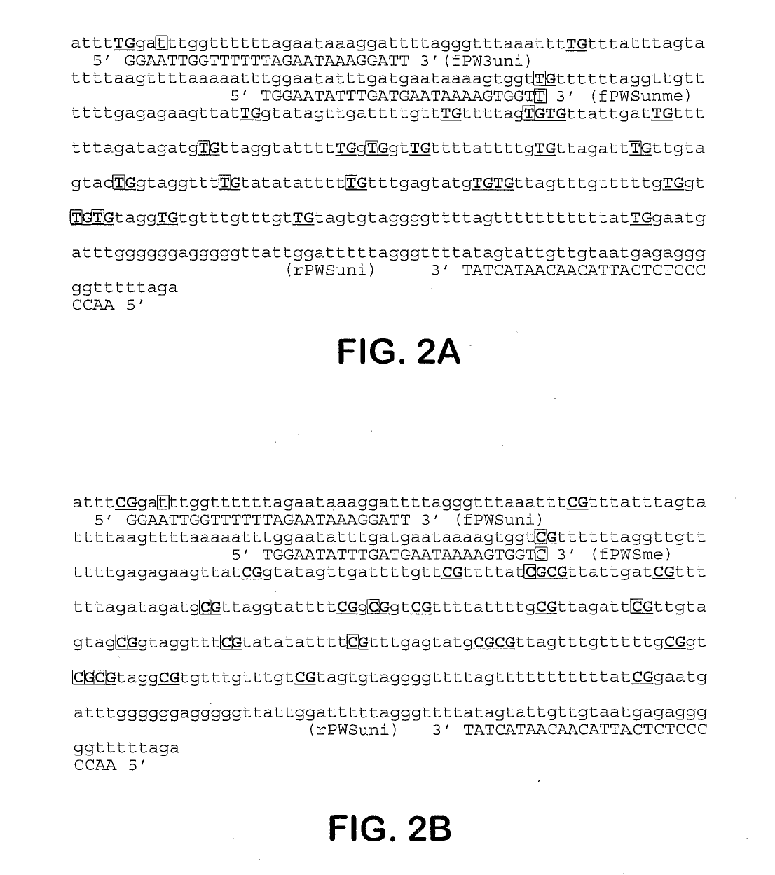

When used in the MSPE assay to detect genomic imprinting disorders, the discrimination primers of the present invention may provide information on the genomic imprinting disorder by identifying the methylation status of the cytosine at the target CpG site. In a preferred embodiment, the discrimination primers may be individually coupled to different 5′ ZipCode sequences that hybridize to cZipCode sequences that are coupled to different colored substrates, preferably, fluorescent microspheres. The methylation status of the extension products is determined by measuring the signal from the different colored substrates.

Where the MSPE assay of the present invention is used to detect PWS or AS, preferred discrimination primers are designed to hybridize to CpG site W in intron 1 of the modified reverse complement strand of the SNRPN gene on the 15q1.2-15q13 chromosomal region. These discrimination primers, which are shown in green in FIGS. 2A and 2B, have the following sequences:

| | |
|---|---|
| 5′-TGGAATATTTGATGAATAAAAGTGGTT-3′ | (SEQ ID NO: 1) |
| 5′-TGGAATATTTGATGAATAAAAGTGGTC-3′ | (SEQ ID NO: 2) |

As shown above and in FIGS. 2A and 2B, the discrimination primers are identical save for the nucleotide at the 3′ end of the primers, which is boxed-in. Discrimination primers designed to hybridize to unmethylated cytosine at the target site of the DNA sample (i.e., CpG site W) will have a thymine at the 3′ end whereas the discrimination primer designed to hybridize to methylated cytosine at the target site in the DNA sample will have a cytosine at the 3′ end. As indicated in FIGS. 2a and 2b, the discrimination primers have been given the shorthand “fPWSunme” and “fPWSme,” which are short for “forward PWS unmethylated primer” and “forward PWS methylated primer,” respectively; SEQ ID NO: 1 represents the sequence for the fPWSunme primer and SEQ ID NO: 2 represents the sequence for the fPWSme primer.

FIGS. 2A and 2B also shows the amplification primers specific for the SNRPN gene that are used to detect PWS or AS with the MSPE assay of the present invention. The SNRPN amplification primers are designed to provide target amplification irrespective of the DNA methylation status at the target site (i.e., CpG site W). The amplification primers, which are identical for both the unmethylated DNA (FIG. 2A) and the methylated DNA (FIG. 2B), have the following sequences:

| | |
|---|---|
| 5′-GGAATTGGTTTTTTAGAATAAAGGATT-3′ | (SEQ ID NO. 3) |
| 3′-TATCATAACAACATTACTCTCCCCCAA-5′ | (SEQ ID NO. 4) |

As shown in FIGS. 2A and 2B, after bisulfite modification, the cytosine of the unmethylated DNA has been converted to thymine (FIG. 2A) whereas the methylated cytosine remains intact (FIG. 2B). FIG. 2A indicates thymine instead of uracil because even though bisulfite treatment initially converts cytosine to uracil, subsequent amplification of the gene product replaces the uracil with thymine. As indicated in FIGS. 2A and 2B, the amplification primers have been given the names “fPWSuni” and “rPWSuni,” which are short for “forward PWS universal primer” and “reverse PWS universal primer,” respectively; SEQ ID NO. 3 represents the sequence for the fPWSuni primer and SEQ ID NO. 4 represents the sequence for the rPWSuni primer.

In the MSPE assay of the present invention, two types of signals may be measured: a dNTP signal and a colored substrate signal. A signal obtained from the labeled dNTPs indicates that an extension product has been created from the discrimination primers. Because an extension product can only be created where the discrimination primer hybridizes to a cytosine or thymine at the target CpG site, a dNTP signal obtained in the MSPE assay confirms the presence of either unmethylated or methylated CpG dinucleotides at the target site of the sample. The signal obtained from the labeled dNTPs allows for the quantification of extended discrimination primers having the cytosine versus the thymine at the methylation site, but does not distinguish the discrimination primers. Whether the discrimination primer binds to methylated or unmethylated DNA is determined by the signal emitted from the colored substrate, e.g., colored beads. In genomic imprinting disorders caused by deletion or functional inactivation of a CpG site on either the maternal or the paternal allele, no extension product (and consequently, no dNTP signals) of the defective allele will result from the MSPE assay because there is no target CpG site for hybridization of the discrimination primer. In this situation, any extension product and colored substrate signal obtained from the MSPE assay will provide information on the methylation status of the CpG site on the allele that is not deleted or functionally inactivated.

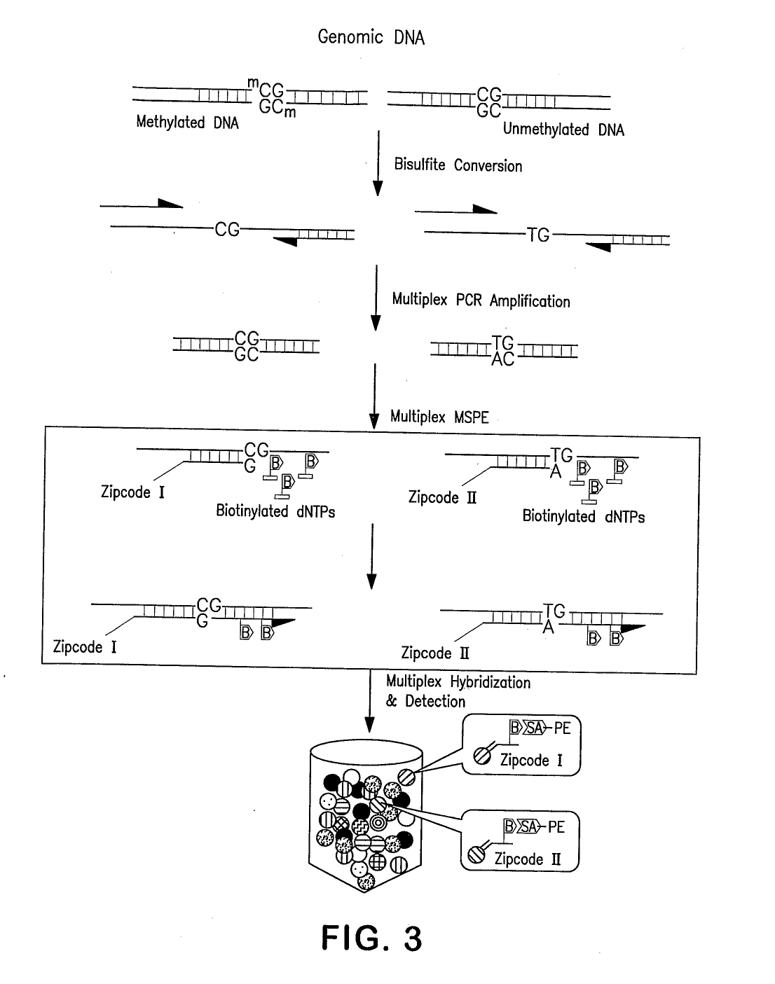

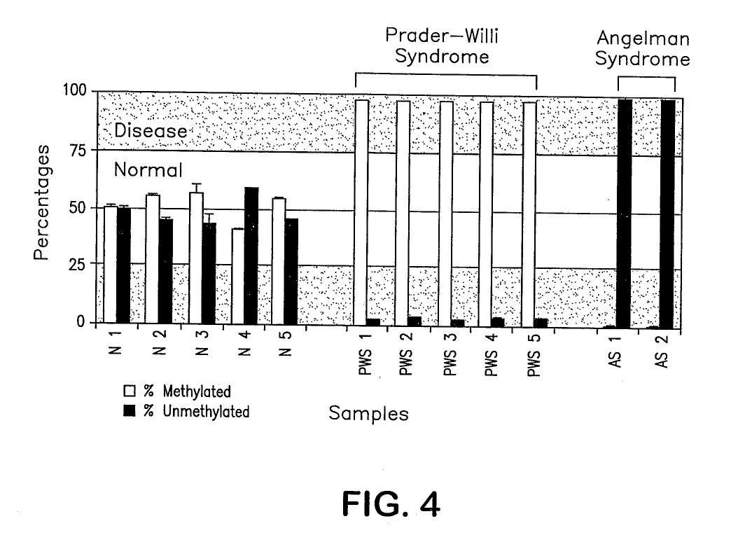

FIG. 3 shows a schematic representation of the MSPE assay of the invention tested on a normal sample that has both parental and maternal CpG sites in intron 1 of the SNRPN gene on 15q11.2-15q13. Generally, normal individuals will have a 50% ratio of methylated to unmethylated cytosines at CpG site W, including both the maternal and paternal alleles of 15q1.2-15q13 (the normal patients in FIG. 4 have a 40% to 60% ratio of methylated to unmethylated cytosines at CpG site W). As previously noted, patients with PWS have deletion or functional inactivation of CpG site W on the paternal allele and patients with AS have deletion or functional inactivation of CpG site W on the maternal allele; consequently, the paternal allele of patients with PWS will not extend during the MSPE assay and the maternal allele of patients with AS will not extend during the MSPE assay. As a result of the non-extension, the disease-state alleles will not contain labeled dNTPs and consequently, will not release a PE (i.e., a labeled dNTP) signal during the MSPE assay.

In yet another embodiment of the invention, there is provided a packaged kit for diagnosing Prader-Willi Syndrome (PWS) or Angelman Syndrome (AS) in a patient, the kit comprising: (a) a compound that when applied to a genomic DNA sample converts unmethylated cytosines to uracil, while leaving methylated cytosines intact; (b) a forward amplification primer and a reverse amplification primer; (c) methylated and unmethylated discrimination primers complementary to a gene segment of a genomic sample with known PWS or AS, wherein both of the discrimination primers are individually coupled to a different 5′ ZipCode sequence, one of the discrimination primers has a 3′ cytosine and the other discrimination primer has a 3′ thymine, and the discrimination primers are designed to hybridize to at least one target CpG dinucleotide of the genomic sample, wherein the at least one CpG dinucleotide is a site associated with PWS or AS; and (d) complementary ZipCode sequences coupled to colored substrates, wherein each unique cZipCode sequence is coupled to a different colored substrate, wherein presence or absence of PWS or AS in the genomic DNA sample is determined by measuring the extension products obtained using the discrimination probes of item (b) and methylation status of the extension products is determined by the colored substrate signals obtained from the cZipCode sequences of item (d).

For the modifying compound, sodium bisulfite may be included in the packaged kit. Within the PWS and AS packaged kit, the forward amplification primer may have SEQ ID NO: 3, the reverse amplification primer may be have SEQ ID NO: 4, the unmethylated discrimination primer may have SEQ ID NO: 1, and the methylated discrimination primer may have SEQ ID NO: 2. When the discrimination probes have SEQ ID NOS: 1 and 2, the at least one target CpG dinucleotide may be CpG site W in intron 1 of the small ribonucleoprotein N(SNRPN) gene on segment q11.2-q13 of chromosome 15. The colored substrates of item (d) of the packaged kit may be colored fluorescent microspheres.

The packaged kit may further comprise DNA polymerases, labeled dNTPs, and buffers. For the DNA polymerase, Taq polymerase is preferably used with the amplification primers and Tsp is preferably used with the discrimination primers. If the labeled dNTPs are biotinylated dNTPs, the kit may further include SA-PE, which will cause visualization of the extension products upon reaction of the biotinylated dNTPs with SA-PE. The buffers included in the packaged kit may include amplification buffers, extension buffers, and hybridization buffers. Example 1 outlines the protocol of the MSPE assay as it is used for the detection of PWS or AS, Example 2 describes one specific assay as it was used on whole blood samples of healthy donors and DNA derived from cells of patients with PWS and AS, and Example 3 shows the results of the MSPE assay applied to normal samples, and PWS samples and AS samples of known disease state. As shown in FIG. 4 and Table 1, normal samples displayed 40% to 60% methylated and unmethylated alleles; PWS samples displayed approximately 96% methylated alleles; and AS samples displayed approximately 99% unmethylated alleles.

As exemplified through the use of the MSPE assay for the detection of PWS or AS, the MSPE assay of the present invention has the advantage of being amenable to a multiplex format; that is, it can be used to identify more than one target site and/or disease state in a single assay. In general use, the MSPE assay is preferably carried out in a high throughput format using microtiter plates having 96 or more wells. An additional advantage of the MSPE assay of the present invention over prior DNA methylation assays is that the diagnostic feature of the MSPE assay may be automated. For example, where a flow cytometer such as the LUMINEX® 100 (Luminex Corp., Austin, Tex.) is used, the proportion values for normal samples may be entered into the system and thus, the comparative data of extension products via PE signal (indicative of cytosine methylation status) for a normal patient versus a PWS or AS patient may be calculated directly by the cytometer, rather than via manual assessment.

For purposes of illustration, the MSPE assay of the present invention has been described for the identification of PWS and AS; however, it is to be understood by one of ordinary skill in the art that the MSPE assay of the present invention is not limited to the detection of these two genomic imprinting disorders and may certainly be extended to detect other genomic imprinting disorders.

While the invention has been described in conjunction with the preferred specific embodiments described herein, the foregoing descriptions as well as the examples that follow are intended to illustrate and not limit the scope of the invention. Other aspects, advantages and modifications within the scope of the invention will be apparent to those skilled in the art to which the invention pertains.

All patents and publications mentioned herein are hereby incorporated by reference in their entireties.

### EXPERIMENTAL

The following examples are put forth so as to provide those of ordinary skill in the art with a complete disclosure and description of how to make and use the compositions of the invention. The examples are intended as non-limiting examples of the invention. Techniques used in the following examples use, unless otherwise indicated, conventional techniques of molecular biology, molecular genetics, and biochemistry that are within the skill in the art. Such techniques are explained fully in the literature. See, for example, Sambrook, et al., MOLECULAR CLONING: A LABORATORY MANUAL, 2^{nd} ed. (1989); OLIGONUCLEOTIDE SYNTHESIS (M. J. Gait, ed., 1984); and NUCLEIC ACID HYBRIDIZATION (B. D. Haines & S. J. Higgins, eds., 1984). Efforts have been made to ensure accuracy with respect to numbers (e.g., amounts, temperature, etc.) but some experimental error and deviations should, of course, be allowed for. Unless indicated otherwise, parts are parts by weight, temperature is degrees centigrade and pressure is at or near atmospheric. All components were obtained commercially unless otherwise indicated.

### Example 1

### The MSPE Assay Protocol for Detection of PWS and/or AS

The MSPE assay of the present invention for the detection of PWS and/or AS is carried out according to the following protocol:

Genomic DNA is obtained from the blood of a patient and is modified with sodium bisulfite to convert 5-unmethylated cytosine at a CpG site to uracil while leaving 5-methylated cytosine at a CpG site unchanged.

PCR amplification is performed on the DNA using the fPWSuni and rPWSuni amplification primers (SEQ ID NOs. 3 and 4; FIGS. 2a and 2b) specific for the bisulfite modified reverse complement strand of the SNRPN gene on the 15q1.2-15q13 chromosomal region. During amplification, the uracil is replaced with thymine.

The fPWSme and fPWSunme discrimination primers (SEQ ID NOs 1 and 2; FIGS. 2a and 2b) specific for CpG site W in intron 1 of the SNRPN gene and coupled at the 5′ end with ZipCode sequences are hybridized to the DNA. The discrimination primers are extended using DNA polymerase in the presence of labeled dNTPs. Since the discrimination primers are only able to extend with a proper match at the last nucleotide, they are able to discriminate between the presence of a cytosine or a thymine at the target CpG site.

Under hybridizing conditions, the sample is exposed to cZipCode sequences coupled to fluorescent microspheres (the 3′ end of the cZipCode sequences are coupled to the microspheres).

The sample is stained with SA:PE.

Using a flow cytometer, such as the LUMINEX® 100 flow cytometer, the extension products derived from a target site containing a cytosine versus a thymine are determined from the PE signal from the labeled dNTPs. Where an extension product is created, the methylation status of the target site, i.e., whether there is a cytosine or a thymine at the methylation site is determined by the fluorescence signal from the microspheres. The data is analyzed manually or where a LUMINEX® 100 flow cytometer is used, automatically to assess if the patient has a disease state based upon the cytosine to thymine proportion to an established cut-off, such as for example, 25% to 75% methylated sites representing a normal patient.

Where the MSPE assay is used in a multiplex format, multiple target sites are analyzed concurrently.

### Example 2

### Application of the MSPE Assay to Whole Blood Samples

Normal genomic DNA was obtained from whole blood using the QIAGEN® QIAAMP® DNA Blood Midi Kit (Qiagen GmbH, Hilden, Germany) according to the manufacturer's protocol. PWS and AS genomic DNA was derived from cells obtained from Coriell Cell Repositories (Coriell Institute for Medical Research, Camden, N.J.). One microgram (μg) of genomic DNA was modified with the EZ DNA Methylation Kit (Zymo Research Corp, Orange, Calif.) according to the manufacturer's protocol.

For the PCR Reaction, 10 μL of sample DNA (from 50 μL eluate obtained from modification of 1 μg DNA) was mixed with the following reaction mix: 100 μM dNTPs; 2 mM MgCl₂; 200 mM of each primer (fPWSuni and rPWSuni); 2 units AMPLITAQ® Gold DNA polymerase (Applied Biosystems, Foster City, Calif.) in a total reaction volume of 100 μL. The PCR Reaction was run under the following conditions: 10 min at 95° C. follows by 40 cycles of 95° C. for 30 seconds, 54° C. for 30 seconds, 72° C. for 30 seconds, and a final extension at 72° C. for 5 minutes. A single amplicon of 419 bp was confirmed on a 3% agarose gel.

For the MSPE assay, 10 μL of 1:10 diluted amplicon was mixed with the following reaction mix: 2.5 mM MgCl₂; 50 nM forward primer specific for the methylated allele (fPWSme); 5 nM forward primer specific for the unmethylated allele (fPWSunme); 7.5 μM dNTPs; 4 μM biotinylated dATP; 0.6 units Tsp DNA polymerase (Invitrogen, Carlsbad, Calif.) in a total reaction volume of 20 μL. The amplicon was extended under the following conditions: 2 min at 95° C. follows by 40 cycles of 94° C. for 30 seconds, 61° C. for 1 minute, 72° C. for 1 minute, and a final extension at 72° C. for 5 minutes.

20 μL of extension product was hybridized using 2500 of each LUMINEX® microsphere (Luminex Corp., Austin, Tex.) as the support in a 50 μL assay. Each extension product contained the following ZipCode sequences at the 5′ end.

Methylated ZipCode Sequence:

(SEQ ID NO. 8)

5′-GACATTCGCGATCGCCGCCCGCTTT-3′-18 spacer-3′-amine
coupled with 3′-amine to microsphere.
Unmethylated ZipCode Sequence:

(SEQ ID NO. 9)

5′-CACCGCCAGCTCGGCTTCGAGTTCG-3′-18 spacer-3′-amine
coupled with 3′-amine to microsphere.

Labeling occurred for 10 minutes at 95° C. and 30 minutes at 50° C. with 0.05 μg SA:PE.

The hybridization product was then washed in a 96-well Multiscreen filterplate (Millipore, Billerica, Mass.) with HIV 3.0 Wash A (Bayer Diagnostics Division, Bayer Healthcare LLC, Berkeley, Calif.); incubated with 50 μL SA:PE (0.05 μg) for 15 minutes, and washed twice with HIV 3.0 Wash A.

For the analysis, the washed hybridization product was resuspended in 80 μL TTL buffer (300 mM Tris-HCl, pH 8; 0.3% Tween 20; 167 mM LiCl) and fluorescence was read on a LUMINEX® 100 flow cytometer.

### Example 3

### Results of the MSPE Assay on Normal, PWS, and as Samples

The methylation status of five normal, 5 PWS, and 2 AS samples of known disease states was investigated in quadruplicate samples using the MSPE assay as described in Examples 1. The average percentage of methylated (M/M+U) and unmethylated (U/M+U) alleles in the samples is shown in the graph at FIG. 4 and Table 1. A range for normals was set at 25% to 75% methylated alleles, consistent with current SNP calling procedures. As shown in the graph at FIG. 4 and as set forth in Table 1, the normal samples ranged between 40% to 60% methylated sites; the PWS samples demonstrated 96% methylated sites; and the AS samples demonstrated 1% methylated and 99% unmethylated sites. The results of this experiment demonstrated that the MSPE assay of the present invention provides excellent discrimination between normal, PWS, and AS samples.

TABLE 1

| | Fluorescence | | | | | | | | | | StdevM | % U | | |
|---|---|---|---|---|---|---|---|---|---|---|---|---|---|---|
| | | | | | Avg | Avg | Stdev | Stdev | % M | Avg % M | M/M + | U/U + | Avg % U | StdevU |
| Sample | M | U | M-Bkg | U-Bkg | M-Bkg | U-Bkg | M-Bkg | U-Bkg | M/M + U | M/M + U | U | M | U/U + M | U/U + M |
| N1 | 282.5 | 291 | 281 | 289.875 | 303.25 | 301.125 | 15.6 | 14.9 | 49.2 | 50.2 | 1.0 | 50.8 | 49.8 | 1.0 |
| | 316 | 307.5 | 314.5 | 306.4 | | | | | 50.7 | | | 49.3 | | |
| | 315 | 321 | 313.5 | 319.9 | | | | | 49.5 | | | 50.5 | | |
| | 305.5 | 289.5 | 304 | 288.4 | | | | | 51.3 | | | 48.7 | | |
| N2 | 418.5 | 350 | 417 | 348.9 | 399.375 | 326.625 | 26.4 | 18.5 | 54.4 | 55.0 | 0.9 | 45.6 | 45.0 | 0.9 |
| | 404.5 | 331 | 403 | 329.9 | | | | | 55.0 | | | 45.0 | | |
| | 362.5 | 305 | 361 | 303.9 | | | | | 54.3 | | | 45.7 | | |
| | 418 | 325 | 416.5 | 323.9 | | | | | 56.3 | | | 43.7 | | |
| N3 | 518 | 370.5 | 516.5 | 369.4 | 532.5 | 406.25 | 36.5 | 46.2 | 58.3 | 56.8 | 3.9 | 41.7 | 43.2 | 3.9 |
| | 579.5 | 394 | 578 | 392.9 | | | | | 59.5 | | | 40.5 | | |
| | 544 | 390 | 542.5 | 388.9 | | | | | 58.2 | | | 41.8 | | |
| | 494.5 | 475 | 493 | 473.9 | | | | | 51.0 | | | 49.0 | | |
| N4 | 179 | 258 | 177.5 | 256.9 | 169.75 | 245.625 | 10.3 | 12.7 | 40.9 | 40.9 | 0.3 | 59.1 | 59.1 | 0.3 |
| | 156.5 | 229 | 155 | 227.9 | | | | | 40.5 | | | 59.5 | | |
| | 177.5 | 253 | 176 | 251.9 | | | | | 41.1 | | | 58.9 | | |
| | 172 | 247 | 170.5 | 245.9 | | | | | 40.9 | | | 59.1 | | |
| N5 | 415 | 344 | 413.5 | 342.9 | 377.25 | 317.375 | 49.3 | 43.1 | 54.7 | 54.3 | 0.4 | 45.3 | 45.7 | 0.4 |
| | 371 | 311 | 369.5 | 309.9 | | | | | 54.4 | | | 45.6 | | |
| | 417 | 358 | 415.5 | 356.9 | | | | | 53.8 | | | 46.2 | | |
| | 312 | 261 | 310.5 | 259.9 | | | | | 54.4 | | | 45.6 | | |
| PWS1 | 799 | 26 | 797.5 | 24.9 | 808.5 | 24.125 | 12.0 | 1.0 | 97.0 | 97.1 | 0.1 | 3.0 | 2.9 | 0.1 |
| | 818 | 26 | 816.5 | 24.9 | | | | | 97.0 | | | 3.0 | | |
| | 800.5 | 24 | 799 | 22.9 | | | | | 97.2 | | | 2.8 | | |
| | 822.5 | 25 | 821 | 23.9 | | | | | 97.2 | | | 2.8 | | |
| PWS2 | 402.5 | 15 | 401 | 13.9 | 468.625 | 17.625 | 47.8 | 2.5 | 96.7 | 96.4 | 0.2 | 3.3 | 3.6 | 0.2 |
| | 514.5 | 20 | 513 | 18.9 | | | | | 96.5 | | | 3.5 | | |
| | 477.5 | 20 | 476 | 18.9 | | | | | 96.2 | | | 3.8 | | |
| | 486 | 20 | 484.5 | 18.9 | | | | | 96.3 | | | 3.7 | | |
| PWS3 | 916 | 29 | 914.5 | 27.9 | 862.5 | 26.125 | 64.8 | 2.1 | 97.0 | 97.1 | 0.1 | 3.0 | 2.9 | 0.1 |
| | 920 | 29 | 918.5 | 27.9 | | | | | 97.1 | | | 2.9 | | |
| | 788.5 | 26 | 787 | 24.9 | | | | | 96.9 | | | 3.1 | | |
| | 831.5 | 25 | 830 | 23.9 | | | | | 97.2 | | | 2.8 | | |
| PWS4 | 493.5 | 21 | 492 | 19.9 | 563.875 | 22.625 | 76.6 | 2.8 | 96.1 | 96.1 | 0.1 | 3.9 | 3.9 | 0.1 |
| | 616.5 | 25 | 615 | 23.9 | | | | | 96.3 | | | 3.7 | | |
| | 645 | 27 | 643.5 | 25.9 | | | | | 96.1 | | | 3.9 | | |
| | 506.5 | 22 | 505 | 20.9 | | | | | 96.0 | | | 4.0 | | |
| PWS5 | 750 | 28.5 | 748.5 | 27.4 | 697.875 | 26.125 | 71.7 | 2.3 | 96.5 | 96.4 | 0.1 | 3.5 | 3.6 | 0.1 |
| | 758.5 | 29 | 757 | 27.9 | | | | | 96.4 | | | 3.6 | | |
| | 685.5 | 27.5 | 684 | 26.4 | | | | | 96.3 | | | 3.7 | | |
| | 603.5 | 24 | 602 | 22.9 | | | | | 96.3 | | | 3.7 | | |
| AS1 | 3 | 148 | 1.5 | 146.9 | 1.25 | 147.125 | 0.5 | 31.1 | 1.0 | 0.8 | 0.3 | 99.0 | 99.2 | 0.3 |
| | 2 | 112 | 0.5 | 110.9 | | | | | 0.4 | | | 99.6 | | |
| | 3 | 188 | 1.5 | 186.9 | | | | | 0.8 | | | 99.2 | | |
| | 3 | 145 | 1.5 | 143.9 | | | | | 1.0 | | | 99.0 | | |
| AS2 | 4 | 193 | 2.5 | 191.9 | 1.75 | 172.625 | 0.5 | 22.9 | 1.3 | 1.0 | 0.2 | 98.7 | 99.0 | 0.2 |
| | 3 | 141.5 | 1.5 | 140.4 | | | | | 1.1 | | | 98.9 | | |
| | 3 | 174 | 1.5 | 172.9 | | | | | 0.9 | | | 99.1 | | |
| | 3 | 186.5 | 1.5 | 185.4 | | | | | 0.8 | | | 99.2 | | |
| PCR | 1 | 1 | −0.5 | −0.1 | 0 | 0 | 0.6 | 0.3 | 80.0 | 87.6 | 32.3 | 20.0 | 12.4 | 32.3 |
| blank | 2 | 1 | 0.5 | −0.1 | | | | | 133.3 | | | −33.3 | | |
| | 2 | 1.5 | 0.5 | 0.4 | | | | | 57.1 | | | 42.9 | | |
| | 1 | 1 | −0.5 | −0.1 | | | | | 80.0 | | | 20.0 | | |
| Luminex | 1 | 1 | −0.5 | −0.1 | −0.25 | 0.125 | 1.0 | 0.5 | 80.0 | 85.5 | 39.9 | 20.0 | 14.5 | 39.9 |
| blank | 2 | 1 | 0.5 | −0.1 | | | | | 133.3 | | | −33.3 | | |
| | 0 | 1 | −1.5 | −0.1 | | | | | 92.3 | | | 7.7 | | |
| | 2 | 2 | 0.5 | 0.9 | | | | | 36.4 | | | 63.6 | | |

We claim:

1. A method for detecting a genomic imprinting disorder in a patient comprising the steps of:
(a) obtaining a sample of genomic DNA from a patient;
(b) modifying the genomic DNA in the sample with a compound that converts unmethylated cytosines to uracil while leaving methylated cytosines intact;
(c) amplifying the DNA from step (b), wherein the uracil is converted to thymine during initial amplification;
(d) hybridizing denatured DNA from step (c) with at least two discrimination primers designed to hybridize to at least one target CpG dinucleotide of the sample, wherein at least one of the discrimination primers has a 3′ cytosine and the other of the at least two discrimination primers has a 3′ thymine;
(e) building extension products from the discrimination primers using DNA polymerase in the presence of labeled dNTPs; and
(f) identifying the DNA based upon signals emitted from the labeled dNTPs, wherein the genomic imprinting disorder is detected by measuring percentage of methylated or unmethylated extension products to total extension products as determined by the dNTP signals.

2. The method of claim 1, wherein the DNA of step (b) is modified with sodium bisulfite.

3. The method of claim 1, wherein the DNA is amplified by PCR.

4. The method of claim 1, wherein the labeled dNTPs are biotinylated dNTPs.

5. The method of claim 1, wherein the extension products from the discrimination primers are visualized upon reaction of the biotinylated dNTPs with streptavidin-phycoerythrin.

6. The method of claim 1, wherein the genomic DNA is from a human patient.

7. The method of claim 6, wherein the target CpG dinucleotide is CpG site W in intron 1 of the small ribonucleoprotein N(SNRPN) gene on segment q11.2-q13 of chromosome 15.

8. The method of claim 7, wherein the genomic imprinting disorder to be determined is Prader-Willi Syndrome.

9. The method of claim 7, wherein the genomic imprinting disorder to be determined is Angelman Syndrome.

10. The method of claim 7, wherein the at least two discrimination primers have SEQ ID NOS: 1 and 2.

11. The method of claim 1, further comprising individually coupling the discrimination primers of step (d) with different 5′ ZipCode sequences.

12. The method of claim 11, wherein prior to step (f), the at least two discrimination primers are hybridized to complementary ZipCode (cZipCodes) sequences coupled to colored substrates, where each unique cZipCode sequence is coupled to a different colored substrate.

13. The method of claim 12, wherein the colored substrates are colored fluorescent microspheres.

14. The method of claim 12, wherein upon creation of an extension product, a signal from the colored substrates indicates methylation status of cytosine at the target CpG dinucleotide.

15. The method of claim 1, wherein the dNTP signals are measured by flow cytometry.

16. The method of claim 14, wherein the dNTP and colored substrate signals are measured by flow cytometry.

17. A method for detecting a genomic imprinting disorder in a patient comprising the steps of:
(a) obtaining a sample of genomic DNA from a patient;
(b) modifying the genomic DNA in the sample with a compound that converts unmethylated cytosines to uracil while leaving methylated cytosines intact;
(c) amplifying the DNA from step (b), wherein the uracil is converted to thymine during initial amplification;
(d) hybridizing denatured DNA from step (c) with at least two discrimination primers designed to hybridize to a target CpG dinucleotide of the sample, wherein at least one of the discrimination primers has a 3′ cytosine and the other of the at least two discrimination primers has a 3′ thymine and further wherein each of the at least two discrimination primers is individually coupled to a different 5′ ZipCode sequence;
(e) building extension products from the discrimination primers using DNA polymerase in the presence of labeled dNTPs;
(f) hybridizing the DNA of step (e) to complementary ZipCode (cZipCode) sequences coupled to colored substrates, wherein each unique cZipCode sequence is coupled to a different colored substrate; and
(g) identifying the DNA based upon signals emitted from the labeled dNTPs and the colored substrates, wherein the genomic imprinting disorder is determined by measuring percentage of methylated or unmethylated extension products to total extension products as determined by the dNTP signals and methylation status of the extension products is determined by the colored substrate signals.

18. The method of claim 17, wherein the DNA of step (b) is modified with sodium bisulfite.

19. The method of claim 17, wherein the DNA is amplified by PCR.

20. The method of claim 17, wherein the colored substrates are colored fluorescent microspheres.

21. The method of claim 17, wherein the labeled dNTPs are biotinylated dNTPs.

22. The method of claim 21, wherein the extension products from the discrimination primers are visualized upon reaction of the biotinylated dNTPs with streptavidin-phycoerythrin.

23. The method of claim 17, wherein the genomic DNA is from a human patient.

24. The method of claim 23, wherein the CpG site is CpG site W in intron 1 of the small ribonucleoprotein N(SNRPN) gene on segment q11.2-q13 of chromosome 15.

25. The method of claim 24, wherein the genomic imprinting disorder to be determined is Prader-Willi Syndrome.

26. The method of claim 24, wherein the genomic imprinting disorder to be determined is Angelman Syndrome.

27. The method of claim 24, wherein the at least two discrimination primers have SEQ IDS NOS. 1 and 2.

28. The method of claim 17, wherein the dNTP and colored substrate signals are measured by flow cytometry.

29. Oligonucleotides for use in a methylation specific primer extension (MSPE) assay comprising at least two discrimination primers complementary to a gene segment of a genomic sample having at least one target CpG site, wherein at least one of the discrimination primers has a 3′ cytosine and the other has a 3′ thymine, wherein extension of the discrimination primer with the 3′ cytosine to the gene segment indicates a methylated cytosine at the CpG site and extension of the discrimination primer with the 3′ thymine to the gene segment indicates an unmethylated cytosine at the CpG site.

30. The oligonucleotides of claim 29, wherein the MSPE assay is used for detecting genomic imprinting disorders.

31. The oligonucleotide of claim 30, wherein the genomic imprinting disorder is determined by the methylation status of the cytosine at the CpG site.

32. The oligonucleotides of claim 29, wherein the discrimination primers are individually labeled with different 5′ ZipCode sequences.

33. The oligonucleotides of claim 32, wherein methylation status of the cytosine at the CpG site is determined by hybridizing the 5′ ZipCode sequences with complementary ZipCode sequences coupled to a colored substrate, wherein each unique cZipCode sequence is coupled to a different colored substrate.

34. The oligonucleotides of 33, wherein the different colored substrates are different colored fluorescent microspheres.

35. A packaged kit for diagnosing Prader-Willi Syndrome (PWS) or Angelman Syndrome (AS) in a patient, the kit comprising:
(a) a compound that when applied to a genomic DNA sample converts unmethylated cytosines to uracil, while leaving methylated cytosines intact;
(b) a forward amplification primer and a reverse amplification primer;
(c) methylated and unmethylated discrimination primers complementary to a gene segment of a genomic sample, wherein both of the discrimination primers are individually coupled to a different 5′ ZipCode sequence, one of the discrimination primers has a 3′ cytosine and the other discrimination primer has a 3′ thymine, and the discrimination primers are designed to hybridize to at least one target CpG dinucleotide of the genomic sample, wherein the at least one CpG dinucleotide is a site associated with PWS or AS; and
(d) complementary ZipCode sequences coupled to colored substrates, wherein each unique cZipCode sequence is coupled to a different colored substrate,
wherein presence or absence of PWS or AS in the genomic DNA sample is determined by measuring percentage of methylated or unmethylated extension products to total extension products obtained using the discrimination probes of item (c) and methylation status of the extension products is determined by the colored substrate signals obtained from the cZipCode sequences of item (d).

36. The packaged kit of claim 35, wherein the compound of item (a) is sodium bisulfite.

37. The packaged kit of claim 35, wherein the forward amplification primer has SEQ ID NO: 3 and the reverse amplification primer has SEQ ID NO: 4.

38. The packaged kit of claim 35, wherein the unmethylated discrimination primer has SEQ ID NO: 1 and the methylated discrimination primer has SEQ ID NO: 2.

39. The packaged kit of claim 35, wherein the at least one target CpG dinucleotide is CpG site W in intron 1 of the small ribonucleoprotein N(SNRPN) gene on segment q11.2-q13 of chromosome 15.

40. The packaged kit of claim 35, wherein the colored substrates of item (d) are colored fluorescent microspheres.

41. The packaged kit of claim 35, further comprising a DNA polymerase, labeled dNTPs, and buffers.

42. The packaged kit of claim 41, wherein the DNA polymerases are comprised of Taq polymerase for use with the amplification primers and Tsp polymerase for use with the discrimination primers.

43. The packaged kit of claim 41, wherein the labeled dNTPs are biotinylated dNTPs.

44. The packaged kit of claim 43, further comprising streptavidin-phycoerythrin (SA-PE), wherein the extension products from the discrimination primers are visualized upon reaction of the biotinylated dNTPs with the SA-PE.

45. The packaged kit of claim 41, wherein the buffers are selected from the group consisting of amplification buffers, extension buffers, and hybridization buffers.
